(19) United States
(12) Plant Patent Application Publication
(10) Pub. No.: US 2006/0107428 P1
(43) Pub. Date: May 18, 2006

(54) Apple tree named '8S6923'

(50) MALUS X DOMESTICA; APPLE

(76) Inventor: W. David Lane, Summerland (CA)

Correspondence Address: Okanagan Plant Improvement Co., Ltd. (PICO), P.O. Box 6000, Summerland, BC, V04 1Z0, CA

(21) Appl. No.: 11/003,323

(22) Filed: Dec. 6, 2004

Related U.S. Application Data
(60) Provisional application No. 60/527,290, filed on Dec. 8, 2003.

(51) Int. Cl. A01H 5/00

(52) U.S. Cl. PLT/172

(57) ABSTRACT

The present invention relates to an apple tree and more particularly to a new and distinct variety broadly characterized by its spurry, productive, precocious and regular bearing tree. The fruit matures under the described conditions approximately the first week of October at Summerland, British Columbia. The fruit is medium-sized, globose to globose conical in shape with yellow skin color, sweet in flavor, and has crisp, firm, and veryjuicy flesh. The variety was developed from a seedling from a controlled cross of the seed parent ‘Splendour’ and the pollen parent ‘Gala’.

### BACKGROUND OF THE INVENTION

### Field of the Invention

Name: ‘8S6923’
Breeders Reference Number ‘8S-69-23’
Genus: Malus
Species: ×domestica Borkh.
Type: Fruiting apple tree
Market Use: Dessert quality apple

This invention relates to apple trees and particularly to a seedling apple tree from a controlled pollination cross made by Dr. W. David Lane of the Pacific Agri-Food Research Centre Summerland apple breeding program located at Summerland, British Columbia, Canada.

The Agriculture and Agri-Food Canada research facility at Summerland was established in 1914. Originally called the Dominion Experimental Farm at Summerland, the name was changed to the Summerland Research Station in 1959, the Summerland Research Centre in 1994 and to the Pacific Agri-Food Research Centre (PARC) Summerland in 1996. The tree fruit breeding program was established in 1924 to provide new varieties for the tree fruit industry of British Columbia, Canada, and the world. The breeding program at Summerland has produced several tree fruit varieties including ‘Spartan’ (unpatented), ‘Silken’ (U.S. Plant Pat. No. 10,740 ), and ‘Creston’ (U.S. Plant Pat. No. 10,739) apples and ‘Van’ (unpatented), ‘Lapins’ (unpatented), and ‘Sweetheart’ (unpatented) sweet cherries. The tree fruit breeders typically produce several thousand seedlings each year.

The objectives of the apple breeding program are: 1) to diversify the product to allow growers to take advantage of niche markets; 2) to improve environmental adaptation to major fruit growing areas, for consistent production of high quality fruit; 3) to reduce the cost of production. The varieties are evaluated for the following traits to insure that the objectives are met.

Upon fruiting, the seedlings are evaluated for fruit and tree quality. Bloom and harvest timing, disease susceptibility and growth habit are evaluated in the field. Promising seedlings are re-propagated by budding or grafting onto rootstocks, and planted out as second test selections in variety evaluation plots. The reproductions are evaluated for varietal stability, disease susceptibility, and fruit and tree quality. The new varieties are compared to reference varieties to establish uniqueness.

The present invention relates to a new and distinct variety of apple tree which was named ‘8S6923’ in 2001. The original cross was made in 1981 and is the result of controlled pollination of a blossom of the seed parent ‘Splendour’ (unpatented) with the pollen of ‘Gala’ (U.S. Plant Pat. No. 3,637 patent expired) made by breeder Dr. W. David Lane.

### SUMARY OF THE INVENTION

‘8S6923’ is a mid-season yellow dessert apple (Malus×domestica Borkh.) with exceptional texture and keeping quality (FIG. 1). The fruit flesh is extremely crisp and juicy, firm and sweet. It retains crispness and juiciness for 6 months in regular air storage at 1° C. The shelf life of fruit after storage is excellent. The tree is highly precocious and productive. The growth habit of ‘8S6923’ is spreading with ample spur development, and moderate vigor. It is not subject to alternate bearing or pre-harvest drop. The stems of adjacent fruit can fuse with the fruit stem and create the appearance of double stems on each fruit.

‘8S6923’ has been tested, under a testing agreement, by several orchardists in BC, as well as by institutional cooperators in the Canadian provinces of Ontario, Nova Scotia, and Quebec.

### Origin

‘8S6923’ resulted from a cross between the seed parent ‘Splendour’ and the pollen parent ‘Gala’ made at the Pacific Agri-Food Research Centre (PARC), Summeriand, BC, by Dr. W. David Lane (See pedigree). Seedlings from this cross were planted in the greenhouse in early 1982, and transplanted to the field nursery in spring of the same year. The seedlings were propagated by budding in place onto Mailing 26 EMLA (unpatented) rootstock in the field in 1984. ‘8S6923’ was selected on the basis of fruit quality and tree habit in 1989, assigned the breeder's number 8S-69-23, and propagated for second test on Mailing 26 EMLA and Mailing 9 EMLA (unpatented) beginning in 1990. The second test trees were planted in randomized, replicated trials with standard cultivars in the spring of 1992 and evaluated from the time of first fruiting for yield, fruit size, fruit quality, storage and sensory quality over 10 years with consistent results. In 1996, the selection was forwarded to elite stage testing under the breeder's test number 8S-69-23

### Distinguishing Characteristics

The variety 8S6923 is a mid season apple maturing about the 2^{nd} of October on average at Summerland, BC, Canada. The fruit has a yellow skin color at maturity and no over color. The fruit is medium is size, globose to globose conical in shape and slightly ribbed. The skin of the fruit is relatively thin, glossy, and shows little russeting on fruit off wood more than one year old. First year fruit can show a high degree of russeting in the stem bowl and the cheeks. The stem of the fruit are of medium length and thickness and can be clubbed and/or hipped. The stems of adjacent fruit can fuse with the fruit stem and create the appearance of double stems on each fruit. The fruit taste is sweet and the flesh is very crisp in texture and very juicy. The fruit storage is very long, trials indicate the fruit will store up to 6 months in air storage at 1° Celsius. The fruit have a long shelf life.

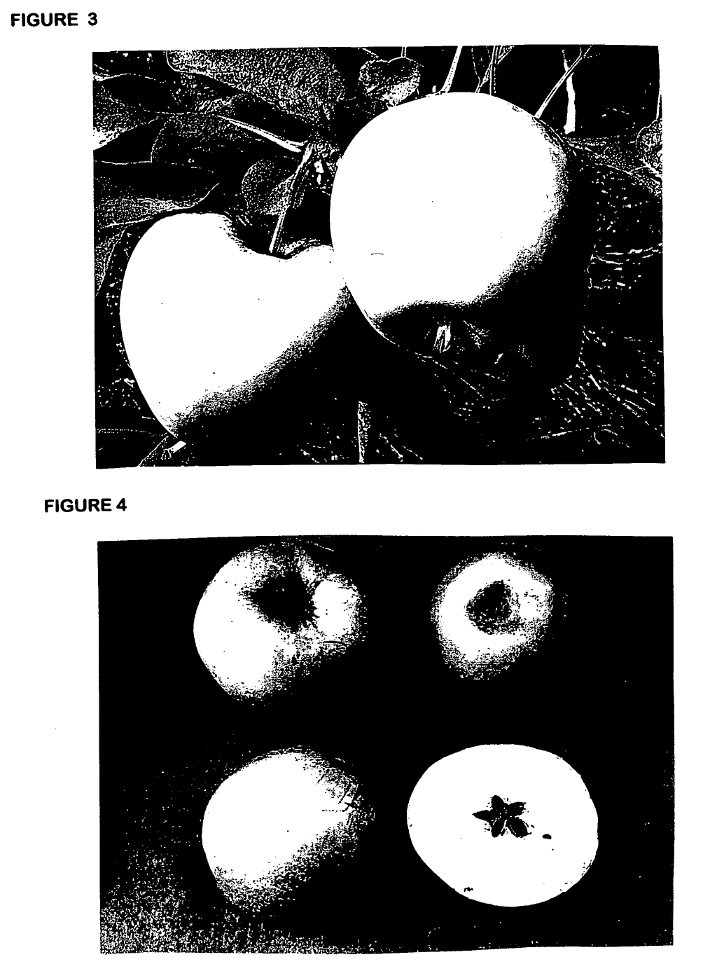

Blind hedonic sensory evaluations of the fruit were run according to Hampson et al. (2000) over 6 harvest years (8 panels) against standard cultivars. In these tests, the appearance of the fruit of ‘8S6923’ was rated significantly higher than ‘Fuji’ (unpatented), as high or significantly higher than ‘McIntosh’ (unpatented), and as high as ‘Golden Delicious’ (unpatented) or ‘Jonagold’ (unpatented). Relative to ‘Royal Gala’ (patent expired) the appearance of ‘8S6923’ was liked as much in 7 panels and significantly less in one. The texture of ‘8S6923’ was liked better than that of ‘Jonagold’, ‘Golden Delicious’ or ‘McIntosh’, better than or equal to ‘Royal Gala’ and equal to ‘Fuji’. The flavor of ‘8S6923’ was liked significantly more than ‘Golden Delicious’ or ‘Mcintosh’, and as well as or significantly more than ‘Fuji’ or ‘Royal Gala’. These blind hedonic tests were run between 55 and 76 days after harvest. In separate blind panels, 12 trained judges evaluated the degree or intensity of various attributes (FIG. 3 and Table 5) relative to standards, as outlined by Hampson et al. (2000). Attribute intensity was rated between 27 and 85 days after harvest of ‘8S6923’, depending on the year.

Shelf life of the fruit of ‘8S6923’ was tested by removing fruit from air storage 117 to 167 days after harvest, leaving it at 20° C. for one week, measuring firmness (Table 3) and then subjecting the fruit to hedonic sensory panels. In 7 hedonic panels (6 harvest years) following the protocol of Hampson et al. (2000), the appearance of ‘8S6923’ was liked as well as or significantly more than ‘Delicious’ (unpatented) and ‘Fuji’, and as well as ‘Royal Gala’, ‘Golden Delicious’, ‘Granny Smith’ (unpatented) and ‘Braeburn’ (unpatented). The texture of the fruit of ‘8S6923’ was liked as well as ‘Braeburn’, as well as or significantly more than ‘Fuji’, and significantly more than ‘Delicious’, ‘Royal Gala’, ‘Granny Smith’ or ‘Golden Delicious.’ The flavor of ‘8S6923’ was liked as well as or significantly more than that of ‘Fuji’, and significantly more than that of ‘Delicious’, ‘Royal Gala’, ‘Granny Smith’, ‘Golden Delicious’ or ‘Braeburn’.

The tree of ‘8S6923’ is very precocious and very productive. The trees have a very heavy bloom and produce very heavy crops annually. The trees produce high quality fruit on spurs and short shoots. The tree produces many spurs.

### BRIEF DESCRIPTIONS OF THE DRAWINGS

The accompanying photographic sheets show various characteristics of the apple variety ‘8S6923’. The colors of the photographs are as nearly true as is possible in a color representation of this type.

In photograph sheet I, FIG. 1, a typical tree is shown. The photograph shows the growth habit a typical tree grown is super spindle style., approximately 4 years old, slightly before optimum maturity of the fruit.

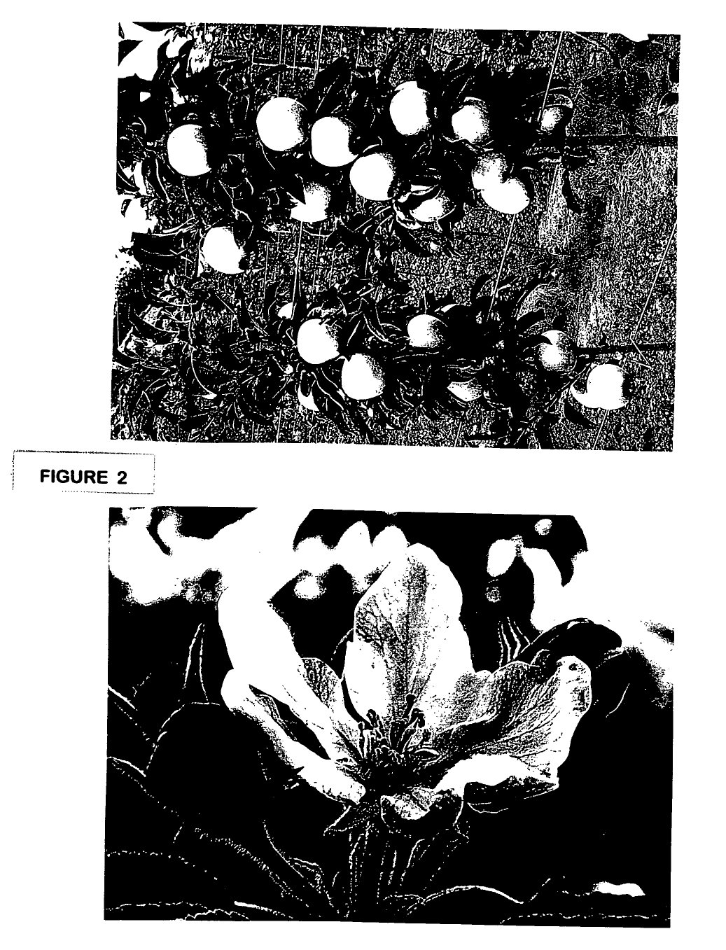

On photograph sheet I, FIG. 2, a typical blossom is shown. The photograph displays the blossoms of ‘8S693’ when the king bloom is fully expanded.

In Photograph sheet II, FIG. 3, typical apples are shown. The fruit shown are close to optimum maturity.

In Photographic Sheet II, FIG. 4, typical apples are shown. The photograph shows typical fruit shape in views from the top, bottom and side. The photograph shows a typical fruit cut transversely exposing the flesh of the fruit and the seed cavity.

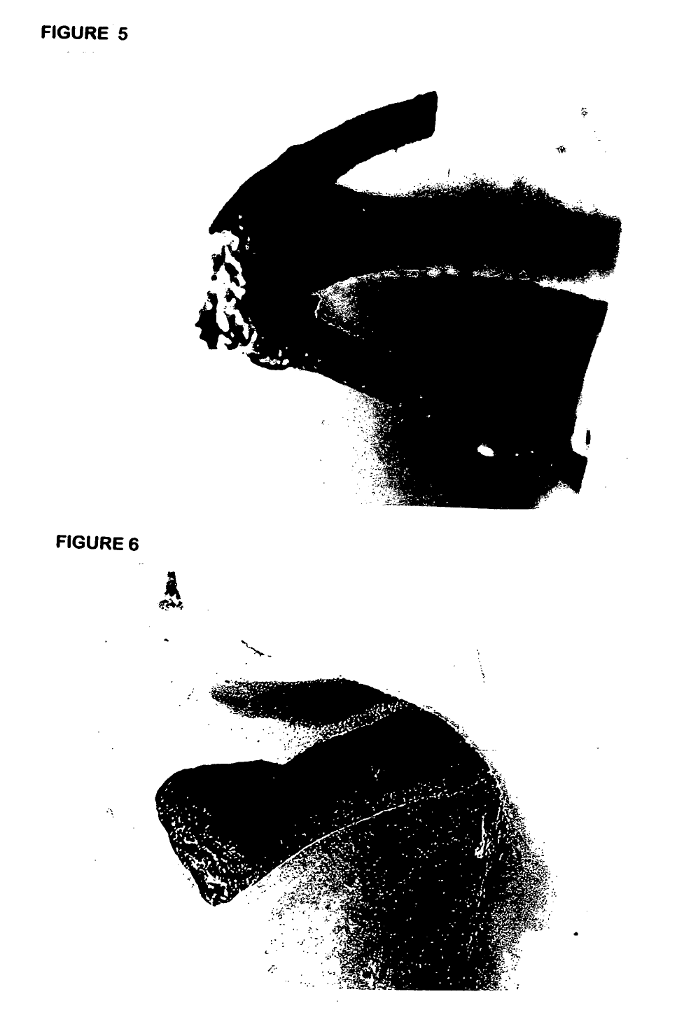

In Photographic Sheet III, FIG. 5, the stem of a fruit is shown. The photograph shows stems that are fused with stems from other fruits.

In Photographic sheet III, FIG. 6, the stem of a fruit is shown. The photograph shows a stem that is clubbed in shape.

### POMOLOGICAL DESCRIPTION

The following description of the flowers, fruit, tree and leaves is based on Zielinski (1955) and the UPOV Malus objective descriptor form (http://www.upov.int/en/publications/tg-rom/tg014/tg_{—}14_{—}8.pdf). Color designations follow those of The Royal Horticultural Society (1966). Measurements are the average of 10 plant parts unless otherwise specified.
Flower:
   Time of 10% king bloom.—mid-season.
   Color of bud at full balloon stage.—RHS 70A/155D. Pedicels green.
   At bloom, color of petals on upper side.—70B/1 55D.
   At bloom color of petals on lower side.—70A/70D (155D when faded).
   Flower type.—single.
   Fragrance.—possesses fragrance.
   Flower size.—Diameter at anthesis, when bloom is pressed flat, 52 mm Individual petals of king bloom 16×24 mm, including claw (mean of 40 petals from 20 flowers).
   Petal shape.—broad elliptic.
   Margins.—overlap at widest point but petals are separate at the bases.
Fruit:
   Shape.—globose to globose conical.
   Shape in cross-section.—round, or sometimes elliptical.
   Ribbing.—weak.
   Side view.—symmetrical or asymmetrical.
   Size.—medium.
   Length.—77 mm (mean of 20).
   Diameter.—83 mm (mean of 20).
   Average fruit weight at harvest.—221 g (See Table 1) Mean weights obtained by counting and weighing all fruit on thinned trees at harvest are slightly smaller, and vary with annual crop load (Table 2).
Skin color:
   Ground color at full maturity.—8C.
   Ground color after storage.—10B.
   Overcolor.—nil.
   Blush some fruit may develop a slight pink-red blush on the cheek.
   Skin.—tender.
   Skin thickness.—thin to medium thickness.
   Skin glossiness.—glossy and smooth at harvest.
   Russet.—may occur on first year fruit.
   Position of russet.—stem bowl and cheeks.
   Wax bloom on skin.—absent.
   Lenticels.—small, round, inconspicuous.
   Lenticel color.—green, except light brown on sunny side.
   Position of lenticels.—more numerous toward calyx end.
   Lenticel russet.—minimal.
   Stem length.—medium (range 21 to 33 mm).
   Stem thickness.—thick to medium thick.
   Stem shape.—clubbed, occasionally hipped. Sometimes stems of adjacent flowers adnate.
   Cavity.—obtuse.
   Depth.—medium.
   Width.—medium.
   Surface.—usually smooth. Occasionally with some greenish gray or tan russet.
Eye basin:
   Depth.—medium.
   Width.—narrow to medium.
   Shape.—sides sloping to flaring, some furrowing.
   Rim.—wavy to smooth.
Calyx:
   Sepals.—persistent, dry, erect and separate at base.
   Curvature.—very slightly recurved.
   Shape.—lanceolate to acuminate.
   Aperture of eye.—closed to half open.
   Eye size.—small to medium.
   Calyx tube.—urn shaped.
   Stamens.—median.
   Core-lines.—clasping.
   Core.—median, open.
   Size.—small to medium.
Carpels:
   Shape in cross section.—round to ovate.
   Shape in longitudinal section.—mucronate to round.
   Symmetry.—symmetrical or axile.
   Inner surface.—smooth, non-tufted.
   Seeds.—Full complement 5 to 9.
   Shape.—acute.
   Seed testa.—non-tufted.
   Color at fruit maturity.—gray brown.
Flesh:
   Texture.—very crisp and breaking.
   Juiciness.—very juicy.
   Graininess.—medium-grained.
   Flesh browning.—cut flesh browns quickly.
   Firmness at harvest.—average 7.16 kg (See Table 1).
   Flavor.—sweet, mild, fresh, delicate, with honey and tropical notes, very good.
   Titratable acidity.—0.69 (See Table 1).
   Soluble solids.—13.8 % (See Table 1).
   Flesh color.—cream to light yellow, 11D.
   Maturity season.—at Summerland, usually harvested in the first week of October, average October 2nd.
   Number of picks.—1 or2.
   Storage.—at least 6 months at 1° C., skin may become slightly waxy after 4 or 5 months.
   Shelf life.—long.
   Use.—dessert apple, appropriate for medium to long term storage.
Tree: Trees of ‘8S6923’ have been tested on M.26 EMLA, M.9 EMLA and Budagovsky 9 (unpatented) rootstocks, with slender spindle and superspindle training systems. ‘8S6923’ has also been propagated successfully on Vineland rootstocks V.1, (unpatented) V.2, (unpatented) V.3 (unpatented) and V.4, (unpatented), but these trees have not yet begun to fruit.
   Vigor.—moderate Trunk-cross sectional area, which is highly correlated with above-ground tree size (Barden and Marini, 2001), is shown in Table 2.
   Shape.—spreading.
   Spurs.—abundant spurs.
   Bearing habit.—mainly on spurs and short shoots, also on one-year-old wood.
   Bearing effiency.—heavy flowering and set is typical, both in terms of number of flowers per cluster and number of clusters.
   Pre-harvest fruit drop.—minimal.
   Precosity.—very precocious.
   Crop.—not subject to alternate bearing.
   Branch angle.—varies from 45 to 90°, mostly 60 to 90°.
   Productivity.—extremely productive. Will overbear without proper thinning. Yield and yield efficiency data appear in Table 2.
   Thinning.—requires prompt and heavy thinning to prevent overbearing and achieve proper fruit size and flavor.
   Dormant first-year shoots.—pubescence weak to medium on upper half.
   Internodes at middle third of shoot.—average 30 mm thickness.
   Lenticel color.—white.
   Lenticel size.—medium.
   Density.—intermediate.
   Bark color on sunny side.—dark brown (RHS 200A).
   Buds.—adpressed.
   Two-year-old wood.—
   Lenticels.—rough.
   Lenticel shape.—oval.
   Lenticel color.—white to pale tan.
   Lenticel size.—up to 1.9×3.5 mm.
   Hardiness.—hardy to at least −20° C. at Summerland. Table 4 shows results of controlled freeze tests in a freeze chamber.
Leaves:
   Growing tip color.—white.
   Cross sectional shape.—concave.
   Pubescence on upper side.—weak.
   Abaxial surface color.—green.
   Anthocyanin coloration.—on apex, margins, petioles and midrib.
Mid-shoot fully expanded leaves:
   Orientation.—upwards and outwards.
   Blade width.—67 mm.
   Blade length.—94 mm.
   Blade length.—width ratio 1.4.
   Apex shape.—cuspidate.
   Leaf margins.—serrate.
   Pubescence on baxial side.—medium.
   Anthocyanin coloration of veins.—medium to strong (RHS 59A).
   Petiole length.—26 mm.
   Stipule length.—11 mm.

### Cultural Notes

‘8S6923’ sets annual heavy crops and must be thinned properly for satisfactory fruit size and taste. Trees are very precocious and will set fruit in the nursery if allowed to do so. On older trees, spur renewal pruning is recommended. If trees are overcropped, the fruit are also hard to pick and therefore more likely to be finger-bruised at harvest. Occasionally, adjacent fruitlets will fuse together.

Some stem end russet often occurs on fruit from the first crop or fruit on one-year-old wood. Older trees have had clean fruit.

The fruit can be harvested in a single pick or two picks. Research to determine the best harvest index is in progress. Skin color is not a good indicator of maturity. We have harvested at starch 4 to 6 on the Blanpied and Silsby (1992) generic starch chart (Table 1) and successfully stored the fruit for up to 7 months in air. No controlled-atmosphere storage research has been done. If picked fully to overmature (iodine staining completely absent), the fruit may develop stem end splits on the tree or in storage. Small spots of superficial scald may appear on some apples if they are highly exposed to the cooling coils in air storage. This can be prevented by covering the fruit with a box of a non-susceptible variety such as ‘Gala’; preliminary observations at PARC show that tarps will also prevent the disorder. Fruit that receive no calcium sprays during the growing season may develop internal browning in long term air storage (8 months or longer), similar to ‘Fuji’. The incidence is higher on when fruit size is very large.

### Virus Testing

‘8S6923’ was tested at the Centre for Plant Health in Sidney, BC using woody and herbaceous host bioindicators, and by serological and molecular methods. No viruses, virus-like agents, viroids, or phytoplasmas were detected. Virus certified trees have been established at the okanagan Plant Improvement Co. Certified budwood orchard at Summerland, BC, Canada.

TABLE 1
Laboratory assessment of fruit quality of ‘8S6923’ at harvest in
different years and different fields. Values are the means of
10-fruit samples, unless otherwise noted.

| | | Harvest | | | | | Titratable |
|---|---|---|---|---|---|---|---|
| | | date | | Firm- | | Soluble | acidity |
| Harvest | | (day of | AFW^{z} | ness | Starch | solids | (% malic |
| year | Field | year) | (grams) | (kg) | Index^{y} | (%) | acid) |
| 1997 | A | 280 | 210 | 7.2 | 5.8 | 14.0 | 0.58 |
| | A | 288 | 227 | 6.6 | 6.1 | 13.3 | 0.65 |
| 1998 | A | 265 | 209 | 7.5 | 6.4 | 13.2 | 0.59 |
| 1999 | A | 278 | 194 | 7.0 | 4.8 | 13.3 | 0.66 |
| | A | 279 | 236 | 6.9 | 4.8 | 13.0 | 0.71 |
| 2000 | A | 271 | 237 | 6.9 | 3.6 | 14.0 | 0.64 |
| | A | 275 | 206 | 7.2 | 4.8 | 13.4 | 0.65 |
| 2001 | A | 276 | 208 | 7.4 | 4.0 | 14.4 | 0.83 |
| | A | 277 | 239 | 7.1 | 3.4 | 14.2 | 0.74 |
| | B | 277 | 231 | 7.0 | 3.6 | 13.4 | 0.72 |
| 2002 | C | 274 | 235 | 7.4 | 4.8^{x} | 13.7 | 0.75 |
| | A | 274 | 219 | 7.3 | 5.1^{x} | 15.1 | 0.63 |
| | B | 275 | 222 | 7.6 | 4.3^{x} | 14.3 | 0.66 |
| Total | | 3589 | 2873 | 93.1 | 61.5 | 179.3 | 8.56 |
| Total | | 276 | 221 | 7.16 | 4.7 | 13.8 | 0.69 |
| Average | | | | | | | |

^{z}AFW, average fruit weight
^{y}Starch index was rated on 5 fruit using the generic 1 to 8 scale of Blanpied and Silsby (1992)
^{x}Mean of 10 fruit

TABLE 2
Average fruit weight (AFW = weight of all fruit on tree/number of
fruit), trunk cross-sectional area (TCSA), cumulative yield and
cumulative yield efficiency (CYE, i.e. cumulative yield divided by
TCSA) of ‘8S6923’ and established cultivars in the same plots
from three randomized, replicated trials on different rootstocks
at Summerland.^{z}

| | | | | | |
|---|---|---|---|---|---|
| | | | | Cumulative | |
| | No. of | AFW | TCSA | yield | CYE |
| Cultivar | trees | (g) | (cm²) | (kg/tree) | (kg/cm²) |

Trial 1: Rootstock M.26 EMLA, 7 years after planting
(5 years of data for average fruit weight)

| | | | | | |
|---|---|---|---|---|---|
| 8S6923 | 4 | 192 b | 13.1 | 63.6 a | 4.80 |
| Braeburn | 4 | 238 a | 9.8 | 37.1 b | 3.79 |
| Golden Delicious | 4 | 243 a | 14.3 | 42.4 b | 3.27 |

Trial 2: Rootstock M.9 EMLA, 6 years after planting (5 cropping years)

| | | | | | |
|---|---|---|---|---|---|
| 8S6923 | 6 | 223 ab | 18.0 | 83.9 a | 5.20 |
| Golden Delicious | 6 | 251 a | 11.9 | 57.4 b | 4.87 |
| Silken | 6 | 197 b | 19.5 | 76.8 ab | 4.26 |

Trial 3: Rootstock Budagovsky 9, 5 years after planting (3 cropping years)

| | | | | | |
|---|---|---|---|---|---|
| 8S6923 | 8 | 211 | 12.8 a | 28.5 a | 2.26 a |
| McIntosh | 8 | 201 | 9.5 b | 13.0 b | 1.38 b |
| Royal Gala | 8 | 207 | 12.0 a | 28.0 a | 2.36 a |
| Silken | 8 | 212 | 12.4 a | 31.1 a | 2.51 a |

^{z}Means within a column and within a trial followed by different letters are significantly different (Fisher's t-test, 5% level)

TABLE 3
Shelf life test data for ‘8S6923’ in different years. The fruit
were removed from 1° C. air storage, and the firmness measured
(Day 0). After one week at 20° C., the firmness was
measured again. Taste panels were run on Day 7.

| | | | | Day | | Day | |
|---|---|---|---|---|---|---|---|
| | | | | 0 | | 7 | |
| | Harvest | | | date | Day 0 | date | Day 7 |
| | date | Harvest | | (day | firm- | (day | firm- |
| Harvest | (day of | firmness^{z} | Days in | of | ness^{z} | of | ness^{y} |
| year | year) | (kg) | storage | year) | (kg) | year) | (kg) |
| 1995 | 268 | 7.6 | 120 | 23 | 7.1 | 30 | 6.6 |
| 1996 | 276 | 7.5 | 123 | 34 | 6.8 | 41 | 6.9 |
| 1997 | 288 | 6.8 | 104 | 27 | 6.7 | 34 | 6.3 |
| 1998 | 265 | 7.3 | 120 | 20 | 6.6 | 27 | 6.9 |
| 1998 | 265 | 7.5 | 133 | 33 | 6.3 | 40 | 6.4 |
| 2001 | 277 | 7.1 | 117 | 29 | 7.4 | 36 | 7.5 |
| 2002 | 274 | 7.4 | 113 | 22 | 6.9 | 29 | 7.4 |

^{z}Mean of 10 fruit
^{y}Mean of 5 fruit. The same fruit were tasted in hedonic panels on Day 7.

TABLE 4
Temperature of incipient damage (TID, the warmest temperature at
which damage occurred to bark or vascular tissue) for twigs in
controlled-freeze tests in two years.^{z} All trees were of the
same age, all were propagated on M.9 EMLA and were in the same field.

| | Number | TID, 1999 | TID, 2000 |
|---|---|---|---|
| Cultivar | of trees | (° C.) | (° C.) |
| 8S6923 | 4 | −23.1 | −22.1 |
| Golden Delicious | 4 | −25.1 | −23.3 |
| Spartan | 4 | −29.8 | −28.3 |

^{z}TID for 8S6923 was not significantly different from Golden Delicious and significantly greater (warmer) than Spartan in both years, according to Dunnett's test.

TABLE 5
Intensity of selected attributes (each rated on a 0 to 9 scale)
of ′8S6923′ fruit relative to commercial cultivars.
Twelve trained judges drawn from a larger pool rated the fruit
in each panel. The values are means weighted inversely
to the error mean square of the analysis of variance for that
taste panel. The number of panels and years that the
cultivar was tested appears in parentheses.
Abbreviations: R. Gala = ′Royal Gala′,
Golden Del. = ′Golden Delicious′.

| | Skin | | | | Flavor | | |
|---|---|---|---|---|---|---|---|
| | tough- | Crisp- | Hard- | Juici- | inten- | Sweet- | Sour- |
| Selection | ness | ness | ness | ness | sity | ness | ness |
| 8S6923 | 4 | 7.2 | 5.8 | 6.7 | 5.3 | 6.2 | 3.8 |
| (10 panels, | | | | | | | |
| 6 yr) | | | | | | | |
| Fuji | 5.3 | 6.2 | 5.8 | 6 | 4.5 | 5.3 | 4 |
| (6 panels, | | | | | | | |
| 5 yr) | | | | | | | |
| R. Gala | 5 | 5.4 | 5.1 | 5.5 | 5 | 5.7 | 3.6 |
| (8 panels, | | | | | | | |
| 6 yr) | | | | | | | |
| Golden Del. | 5.5 | 5.4 | 3.3 | 4.6 | 4.8 | 5 | 4.4 |
| (2 panels, | | | | | | | |
| 2 yr) | | | | | | | |

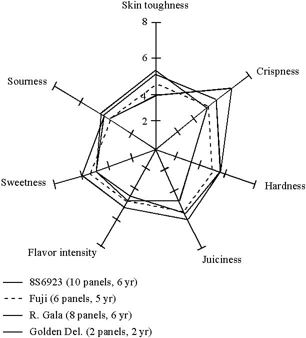

### Pedigree of the Apple Tree Named 8S6923

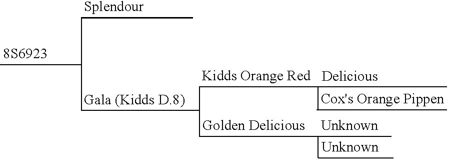

We claim:

1. A new and distinct variety of apple tree, originating from a controlled cross of ‘Splendour’×‘Gala’ substantially illustrated and described, which is most similar to ‘Golden Delicious’ and characterized as to novelty by the yellow skin color, bright luster, globose to globose conical shape, frequent fusing of the fruit stem and the unique combination of taste, texture and juiciness which are different from other dessert quality apples.
